(19) United States
(12) Patent Application Publication
(10) Pub. No.: US 2003/0064409 A1
(43) Pub. Date: Apr. 3, 2003

(54) Substrate composition for multispectural imaging

(76) Inventor: Hicham Fenniri, West Lafayette, IN (US)

Correspondence Address: BARNES & THORNBURG, 11 SOUTH MERIDIAN, INDIANAPOLIS, IN, 46204

(21) Appl. No.: 10/112,830

(22) Filed: Mar. 29, 2002

Related U.S. Application Data
(60) Provisional application No. 60/280,031, filed on Mar. 30, 2001.

(51) Int. Cl. G01N 33/53; C08F 16/10

(52) U.S. Cl. 435/7.1; 436/164; 526/320

(57) ABSTRACT

Polymeric resins having unique spectral fingerprints are provided. The unique spectral fingerprints are produced by varying the co-monomers used to synthesize the resins. Each co-monomer has distinct spectral properties that are additive to produce the unique spectral fingerprints of the polymeric resins. Methods are also provided for use of the polymeric resins in self-deconvolution of combinatorial libraries.

### CROSS-REFERENCE TO RELATED APPLICATION

This application claims the benefit of U.S. application serial No. 60/280,031 filed Mar. 30, 2001.

### FIELD OF THE INVENTION

The present invention relates generally to resin supports for chemical synthesis. Specifically the invention relates to resin supports having unique spectral fingerprints.

### BACKGROUND OF THE INVENTION

Resin-supported combinatorial libraries generated through split synthesis can be screened using encoding or deconvolution methods. The latter strategy proved to be extremely effective in identifying active members from small and large soluble or resin-supported libraries. Although they all derive their roots from Houghten's original schemes, deconvolution strategies can be classified into five families, dual-defined scanning, positional scanning, indexed libraries, recursive deconvolutions and deletion synthesis deconvolution methods.

However, these encoding and deconvolutive techniques tend to be time consuming and expensive which increases the cost of developing useful compounds. Accordingly, an efficient strategy for the structural elucidation of active members of combinatoral libraries is desirable.

### SUMMARY OF THE INVENTION

Polymeric resins having unique spectral fingerprints are provided. In one aspect of the present invention, the unique spectral fingerprints of the polymeric resins are determined by routine spectral methods such as, but not limited to, infared (IR) and Raman spectroscopy. The resulting spectra of the resins, or the spectral fingerprint, are converted to barcode format so that the identity of the resins may be readily determined. The barcodes can be identified either visually or by standard data processing programs.

In another aspect of the invention the polymeric resins are synthesized from co-monomers. The co-monomers used each have distinct spectral fingerprints which, when combined in a polymeric resin of the present invention, are additive to produce a resin having a unique spectral fingerprint. Therefore the unique spectral fingerprint of the polymeric resin is determined by the number of co-monomers in the polymeric resin as well as the amount of the co-monomers. The polymeric resin of the present invention has at least one co-monomer unit. The number of co-monomers that can be used to synthesize a polymeric resin will be limited only by the strength of the spectral signal of the monomer. It will be appreciated that the stronger the spectral signal of a co-monomer, the lower the amount of the co-monomer required as a percentage of the total co-mononers in the polymeric resin to contribute to the polymeric resins spectral fingerprint. Therefore, the less the percentage of a co-monomer required, the greater the number of co-monomers that can be used to synthesize a polymeric resin of the present invention.

In a further aspect of the present invention, the polymeric resins are used in combinatorial synthesis and screening the resulting combinatorial libraries. For example, the polymeric resins of the present invention can be used as support beads in dual recursive deconvolution (DRED) for self-deconvolution of combinatorial libraries. The first building blocks of the combinatorial library are covalently attached to the polymeric resins by chemical linkers. Non-limiting examples of such building blocks are amino acids, nucleic acids and chemical molecules and compounds. Each distinct building block is attached to a spectoscopically distinguishable resin, so that the building block in the first position can be identified by identifying the polymeric resin. For example, if all 20 naturally occuring amino acids are to be used as building blocks, then 20 spectoscopically distinct polmeric resins would be required.

In another aspect of the present inventiona a method of determining the structure of a compound which is bound to a solid support matrix is disclosed. The method includes (a) subjecting the solid support matrix to a spectroscopic technique so as to generate spectrographic data of the solid support matrix, (b) determining a chemical composition of the solid support matrix based upon the spectrographic data generated, and (c) determining the chemical identity of a building block of the compound based upon the chemical composition of the solid support matrix.

Additional objects, advantages, and features of the present invention will become apparent from the following description, taken in conjunction with the accompanying drawings and appended claims.

### BRIEF DESCRIPTION OF THE DRAWINGS

The various advantages of the present invention will become apparent to one skilled in the art by reading the following specification and by referencing the following drawings in which:

FIG. 1A is a schematic illustrating the suspension co-polymerization procedure for the preparation of beaded polymeric resins;

FIG. 1B is a schematic showing the structures of examples of styrene derivative co-monomers used in polymeric resins;

FIG. 2A is a photograph showing the white light image of a randomly selected area of the DRED beads mixture positioned in the NIRIM's field of view;

FIG. 2B shows the specific Raman imaging of DRED bead #10232;

FIG. 2C shows the specific Raman imaging of DRED bead #11011;

FIG. 2D shows the specific Raman imaging of DRED bead #10131;

FIG. 3 is a scanninng electron microscopy micrograph of the DRED beads showing their spherical, smooth and monodisperse nature;

FIG. 4 is an atomic force microscopy image of the surface of the DRED beads;

FIG. 5A is an IR spectra of polystyrene (bead #10262), poly(4-methylstyrene) (bead #10241), and poly(styrene-co-4-methylstyrene) (bead #10131) demonstrating the additive nature of the spectra;

FIG. 5B is a Raman spectra of polystyrene (bead #10262), poly(4-methylstyrene) (bead #10241), and poly(styrene-co-4-methylstyrene) (bead #10131) demonstrating the additive nature of the spectra;

FIG. 6 depicts bead size distribution of barcoaded resins;

FIG. 7 is a histogram illustrating results obtained with PVA in the absence and present of DBS;

FIG. 8 illustrates the effect of stirring speed on bead size distribution and overall yield of beaded materia;

FIG. 9. is a table of data illustrating chloromethylstyrene incorporation quantified by potentiometric titration of the resins' chloride content;

FIG. 10 depicts FTIR spectra of (1) bead #10241, (2) bead #10241 and linker, (3) bead #10241, linker, and Fmoc-Gly, and (4) bead #10241, linker, and Fmoc-Phe; and

FIG. 11 depicts Raman spectra of (1) bead #10241, (2) bead #10241 and linker, (3) bead #10241, linker, and Fmoc-Gly, and (4) bead #10241, linker, and Fmoc-Phe.

### DETAILED DESCRIPTION OF THE INVENTION

Polymeric resins having unique spectral fingerprints are provided. In one aspect of the present invention, the unique spectral fingerprints of the polymeric resins are determined by spectoscopic methods such as, but not limited to, infared (IR) and Raman spectroscopy. In the present invention, spectral fingerprints are meant to include the spectra obtained from a spectroscopic method. The resulting spectra of the resins can further be converted to barcode format so that the identity of the resins may be readily determined. The barcodes can be identified either visually or by standard data processing programs. For example, the barcodes can be decoded using standard laser barcode readers or with commercial data analysis and pattern recognition software packages.

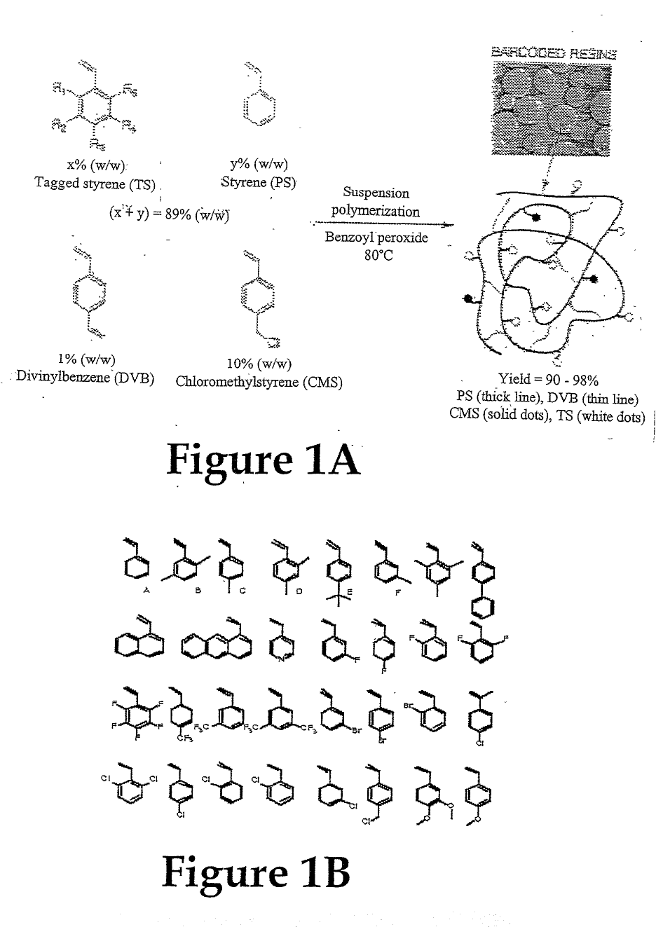

The polymeric resins of the present invention are made up of at least one co-monomer having a unique signature spectra which, when used to form a polymeric resin of the present invention, produce a resin having a unique spectral fingerprint. Non-limiting examples of commercially available styrene co-monomers that can be used in the present invention are shown in FIG. 1B. However, any co-monomer may be used that has a distinct signature spectra and is amenable to forming a polymeric resin. Furthermore the co-monomers, once incorporated into the resin, preferrably are chemically inert. The number of different co-monomers that can be used to synthesize a polymeric resin will be limited only by the strength of the spectral signal of the monomer. The stronger the spectral signal of a co-monomer, the lower the amount of the co-monomer required as a weight percentage of the total co-monomers in the polymeric resin for the monomer to contribute to the polymeric resin's spectral fingerprint. It will be appreciated therefore, that the less the percentage of a co-monomer required, the greater the number of co-monomers that can be used to synthesize a polymeric resin of the present invention. By way of non-limiting example, polymeric resins formulated from co-monomers A-F of FIG. 1B required that a co-monomer added to a polymerization mixture when synthesizing the polymeric resins be greater than about 10-20% (w/w) as compared to the total weight of the co-monomers. When added in amounts less than about 10-20% (w/w), the co-monomers A-F did not display strong and unique spectral signals and the spectrm was dominated by the main co-monomer(s).

The ratio, based on weight, of the co-monomers to each other in the polymeric resin can also vary. For example, a polymeric resin having three co-monomers of similar spectral signal strength may have a co-monomer ratio of 1:1:1 (w/w). However, if one of the co-monomers has a stronger spectral signal than the other two, the optimal ration of co-monomers may be 1:2:2 (w/w). Factors affecting the strength of the spectral signal of a co-monomer may be the chemical structure of the co-monomer itself as well as the sensitivity of the spectroscopic method used.

Non-limiting examples of 24 polymeric resins formulated using styrene-based co-monomers A-F from FIG. 1B are shown in Table 1.

[image]

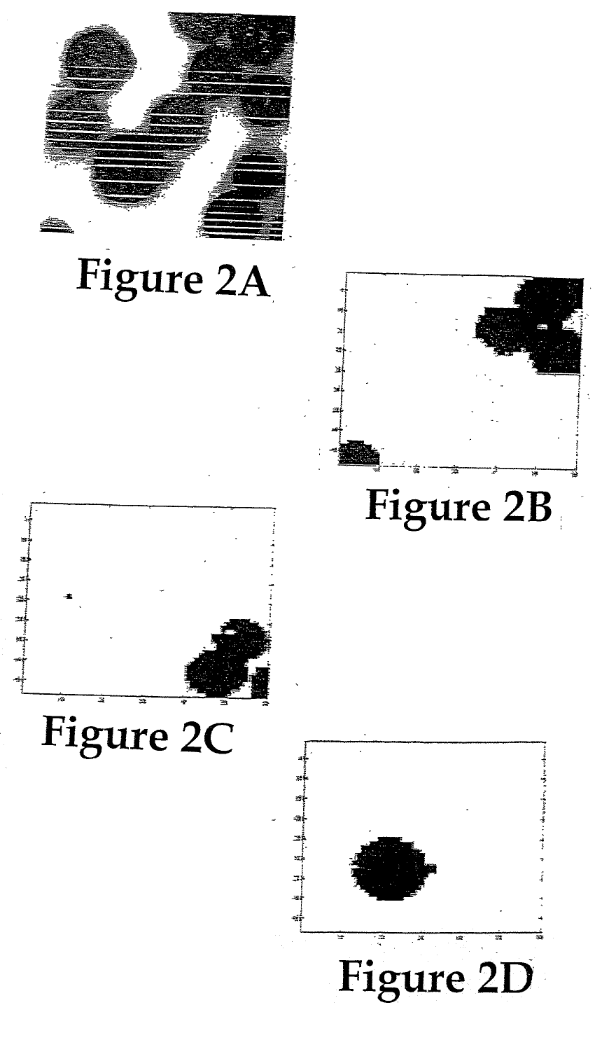

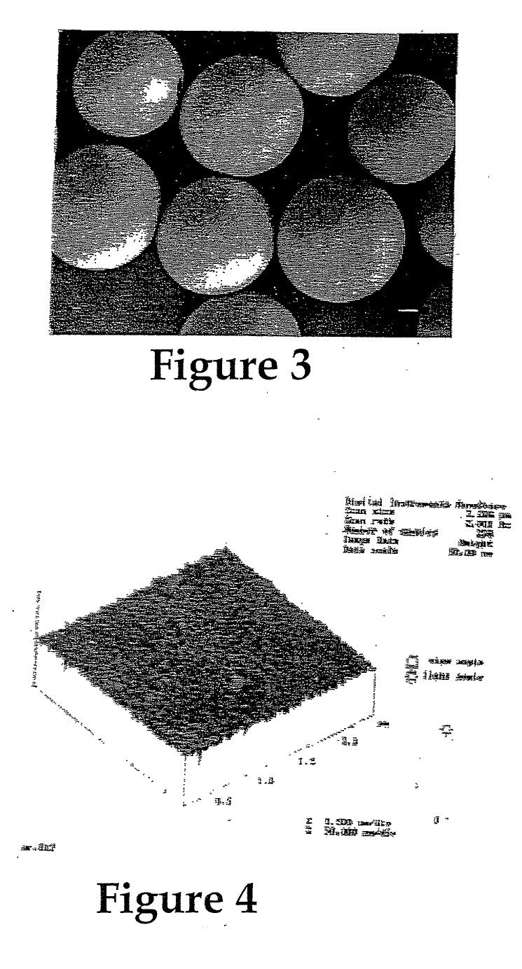

Co-monomers A-F are commonly known as styrene, 2,5-dimethylstyrene, 4-methylstyrene, 2,4-dimethylstyrene, 4-tert-butylstyrene and 3-methylstyrene. To simplify data processing and analysis, the characteristic IR and Raman spectral features of each resin between 400 and 2000 cm⁻¹ was converted into unique barcodes readily identified with standard laser-based bar code readers. The polymeric resins can be readily identified from a mixture of the 24 spectroscopically distinct beads of Table 1 using the barcodes defined in the Table (FIGS. 2B-2E). The Near Infrared Raman Imaging (NIRIM) instrument utilized in this study uses fiber bundle image compression (FIC) technology to simultaneously collect a 3-D Raman spectral imaging data cube (λ-x-y) containing an optical spectrum (λ) at each spatial location (x-y) of a globally illuminated area. It should be noted that this is a real-time imaging technique as opposed to the previously reported step—scan methods, which require longer time to generate an image of the sample.

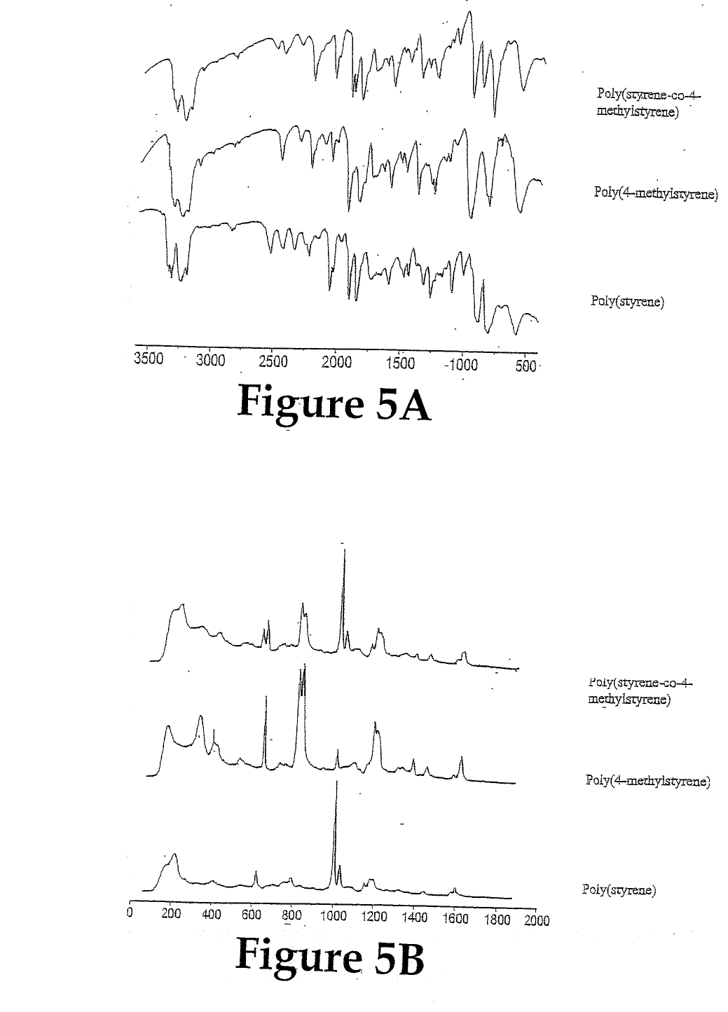

The polymeric resins of the present invention composed of two or more co-monomers may exhibit spectra that are additive, i.e., the sum of the spectra of the corresponding homo-polymers. For example, the Raman spectrum of poly(styrene-co-4-methylstyrene) is the sum of the Raman spectra of polystyrene and poly-4-methylstyrene (FIG. 5B). The same result was observed using IR spectroscopy (FIG. 5A). Therefore, the spectral fingerprint and the resulting barcode of a polymeric resin can be predicted based on the individual spectra of the co-monomers.

The co-monomers incorporated into the polymeric resins of the present invention may be substituted co-monomers. The substituted co-monomers may be alkylated co-monomers or heteroatom-containing co-polymers. Heteroatom-containing co-monomers may alter the chimical properties of the beads significantly, particularly the swelling of the polymeric resins in various solvents. They are more limited than alkylated co-monomers in their solvent compatability. For example, halogenated co-monomers produce polymeric resins that swell much more in chlorinated solvents while oxygen-containing co-monomers yield polymers with the widest spectrum of solvent compatibility, ranging from apolar solvents such as toluene to polar solvents like methanol. Care must also be taken that the heteroatom-containing co-monomers are inert when introduced into the polymeric resin.

The polymeric resins of the present invention can be synthesized by methods known in the art for making polymeric resins. For example, co-monomers can be randomly introduced into the structure of a polymeric resin using standard suspension co-polymerization techniques (FIG. 1A). Preferably the polymeric resins are formed in a single suspension co-polymerization step using Arshady's reactor. Arshady, R., J. Macromol. Struct. Rev. Macromol. Chem. Phys. C32:101 (1992); Arshady, R. et al., React. Polym. 1: 159 (1983); Kempe, M. et al., J. Am. Chem. Soc. 118:7083 (1996). A stabilizer, preferably a water-stable polymer, may also be added to the polymerization suspension. The addition of a stabilizer prohibits the formation and subsequent aggregation of microbeads during the polymerization. Alternatively, bead size distribution may be controlled by controlling the rate of mixing (i.e., stirring speed) during the reaction. Preferably the rate of mixing is between 300-600 rpm.

In another aspect of the invention, methods for using the polymeric resins in self-deconvolution of combinatorial libraries are provided. The polymeric resins are used as support beads for the synthesis of the combinatorial libraries. Dual recursive deconvolution (DRED) is a hybrid of deconvolution and encoded resin strategies for screening the resulting libraries. DRED operates through the iterative identification of the first and last randomized postions of active members of combinatorial libraries generated through split synthesis. Fenniri, H. et al., Angew. Chem. Int. Ed. 39:4483 (2000). The last building block can be readily obtained from pool screening after the last step of the split synthesis, while the first position can be “encoded” by the unique spectral fingerprint of the polymeric resins of the present invention. The first building block is covalently attached to a polymeric resin having a unique spectral fingerprint. Each different building block is attached to a different polymeric resin with a different spectral fingerprint. For example, if the building blocks are the 20 naturally occurring amino acids, then 20 different polymeric resins are required, one for each of the 20 amino acids.

The polymeric resins of the present invention may include a small amount of a reactive co-monomer for attaching a building block. The amount of the reactive co-polymer is sufficient for attachment of the building block, but low enough that it does not effect the spectral fingerprint of the polymeric resin. Concentrations of about 5 mMol/g of resin may be sufficient. For example, chloromethylstyrene was incorporated in the polymeric resin at a level proportional to its molar ratio in the polymerization mixture and can be modulated to a final loading of 0.2 to 1.2 mMol/g resin.

The first building blocks for the combinatorial library may be attached directly to the polymeric resin or may be attached through a linker. The linker can be attached to the reactive co-monomer of the polymeric resin and then the building block attached to the linker. The linker is chosen such that it does not alter the unique spectral fingerprint of the polymeric resin. For example, polymeric resin beads incorporating chloromethylstyrene were functionalized with a the Wang linker, 4-hydroxymethylphenol. In most cases, a single coupling was sufficient to react with all of the reactive co-monomer. When necessary, a second coupling was sufficient to cover the unreacted co-monomer. Amino acid building blocks, Gly, Ala and Phe were attached to the linker by loading Fmoc-gly, Fmoc-Ala and Fmoc-Phe onto the resins using standard peptide chemistry. Other linkers and the chemistry required to attache other building blocks are known in the art.

The chemical and physical properties of the polymeric resins can be tailored to the reaction and assay conditions of the combinatorial library. Swelling properties can particularly be tailored to the reaction and assay conditions. Swellability of a resin in a given volume is a multifaceted property reflecting the chemical structure of the polymer backbone, degree of cross-linking and the architecture of the polymer matrix. The 3D structure of the polymer network takes shape according to the conditions prevailing during the formation of the beads. A number of general conclusions can be drawn from the swelling patterns in different solvents. For instance, the extent of polymer swelling decreases as the degree of cross-linking increases. Swellability will also be determined by polymer-solvent compatibility, which is determined by the chemical structure of the polymer backbone. This is particularly relevant in any discussion of site-accessibility of polymer-bound reactive sites. In the present case, the lightly cross-linked microporous spherical beads are expected to behave very much like Merrifield resin (commonly used as a solid support for synthesis) and, hence, the extent of swelling reflects the solubility parameters, that is, the closer the solubility parameters of the polymer and the solvent, the greater the extent of polymer swelling.

The swelling properties of the polymeric resins were found to be very dependent on the co-monomers used. Halogenated monomers lead to polymers that swell much more in chlorinated solvents while oxygen-containing monomers yield polymers with the widest spectrum of solvent compatibility, ranging from apolar solvents such as toluene to polar solvents like methanol. Alkylated monomers yield polymers with swelling properties similar to Merrifield resin. Table 2 summarizes the swelling properties of the 24 polymeric resin examples of Table 1. Table 2. swelling properties of the DRED beads dimethylsulfoxide (DMSO); tetrahydrofirane (THE), toluene (Tol), N,N-dimethylformamide (EME), ethanol (EtOH), N,N-dimethylacetamide (DMA), dichloromethane (DON), and 1,4-dioxane (DOX).

Swellability (mL/g)

| Ref No. | DMSO | THF | Tol | EtOH | DMF | DMA | DCM | DOX |
|---|---|---|---|---|---|---|---|---|
| 10262 | 0.8 | 7.9 | 7.1 | 1.8 | 4.9 | 6.0 | 7.4 | 6.9 |
| 10312 | 0.9 | 6.6 | 6.2 | 2.4 | 3.1 | 4.3 | 5.2 | 4.3 |
| 11012 | 0.9 | 4.2 | 4.7 | 2.3 | 1.5 | 3.3 | 4.7 | 4.4 |
| 11031 | 1.0 | 5.6 | 5.4 | 3.2 | 1.3 | 2.9 | 5.0 | 4.3 |
| 11032 | 1.1 | 3.6 | 4.3 | 2.7 | 1.4 | 2.9 | 4.9 | 3.9 |
| 11022 | 0.8 | 3.7 | 4.7 | 2.2 | 1.5 | 3.1 | 4.4 | 3.6 |
| 10311 | 1.0 | 4.9 | 4.8 | 2.6 | 1.6 | 3.3 | 4.5 | 3.8 |
| 11214 | 0.8 | 4.3 | 4.2 | 2.5 | 1.6 | 2.7 | 4.2 | 3.9 |
| 11011 | 1.1 | 5.6 | 5.7 | 3.4 | 1.5 | 3.9 | 5.0 | 4.2 |
| 10241 | 0.8 | 6.5 | 6.1 | 3.5 | 2.0 | 4.7 | 5.7 | 5.1 |
| 10191 | 1.1 | 4.8 | 4.9 | 2.2 | 1.7 | 3.4 | 4.7 | 4.3 |
| 10242 | 0.8 | 5.0 | 5.2 | 2.3 | 1.5 | 2.9 | 4.4 | 4.0 |
| 10131 | 1.0 | 5.4 | 5.4 | 2.5 | 1.4 | 4.3 | 5.1 | 4.7 |
| 10142 | 0.8 | 5.4 | 5.3 | 2.7 | 1.9 | 3.2 | 4.9 | 4.3 |
| 10232 | 1.0 | 4.6 | 4.8 | 2.3 | 1.4 | 2.6 | 4.3 | 4.0 |
| 10181 | 1.1 | 5.5 | 5.4 | 2.6 | 1.6 | 3.6 | 4.9 | 4.5 |
| 11021 | 0.9 | 5.5 | 5.3 | 2.7 | 1.6 | 3.2 | 4.9 | 4.6 |
| 10132 | 0.9 | 5.7 | 5.3 | 2.7 | 1.6 | 3.5 | 5.1 | 4.7 |
| 10172 | 1.2 | 5.2 | 5.1 | 2.4 | 1.5 | 2.6 | 4.6 | 4.1 |
| 10202 | 0.9 | 5.8 | 5.7 | 2.9 | 1.6 | 3.6 | 5.0 | 4.4 |
| 10192 | 0.9 | 5.1 | 5.3 | 2.3 | 1.4 | 3.0 | 4.8 | 4.3 |
| 10122 | 1.0 | 4.8 | 5.0 | 4.4 | 1.3 | 3.0 | 4.7 | 4.2 |
| 10212 | 0.9 | 4.8 | 4.9 | 2.3 | 1.3 | 2.8 | 4.3 | 3.8 |
| 10141 | 0.9 | 5.4 | 5.2 | 2.4 | 1.6 | 3.7 | 5.1 | 4.8 |

After attaching the initial building block to the polymeric resin, a combinatorial library is generated. The library is then screened by subjecting the polymeric resin support to a spectroscopic technique to generate spectral data and identifying the first building blocks based on the spectral data of the polymeric resin.

Materials.

Benzoyl peroxide (BPO), poly(vinylalcohol) (PVA), sodium dodecylbenzene sulfonate (DBS), 80% divinylbenzene (DVB), and all the monomers were purchased from Aldrich. The co-monomers were distilled under reduced pressure to remove the stabilizers and then stored at +4° C. Wang linker, 4-(hydroxymethyl) phenol, was crystallized from distilled water and dried under high vacuum before use. Anhydrous N,N-dimethylacetamide (DMA), sodium methoxide and all the other reagents were used as received from Aldrich. The other solvents: N,N-dimethylformamide (DMF), dichloromethane (DCM), 1,4-dioxane, toluene, ethanol, tetrahydrofuran (THF), and dimethylsulfoxide (DMSO) were distilled prior to use. An IKA-RW20 mechanical motor was used to maintain and control the stirring speed of the suspension polymerization reaction. The reaction vessels and impellers were designed according to Arshady, R., Ledwith, A. React. Polym. 1983, 159-174. In a few cases a standard Morton flask (ChemGlass) was used as a reactor. FTIR spectra were recorded on a Perkin Elmer 2000 FTIR spectrometer. The beads and KBr were thoroughly mixed and the mixture was pressed to form a pellet, then the spectra were recorded. Single bead FTIR was performed according to procedures described by Yan, B., et al. J. Comb. Chem. 2001, 3, 78-84. Single bead Raman spectra were recorded on a Raman micro-imaging system as described in Fenniri, H., et al., Angew. Chem. Int. Ed. 2000, 39, 4483-4485.

Suspension Polymerizations.

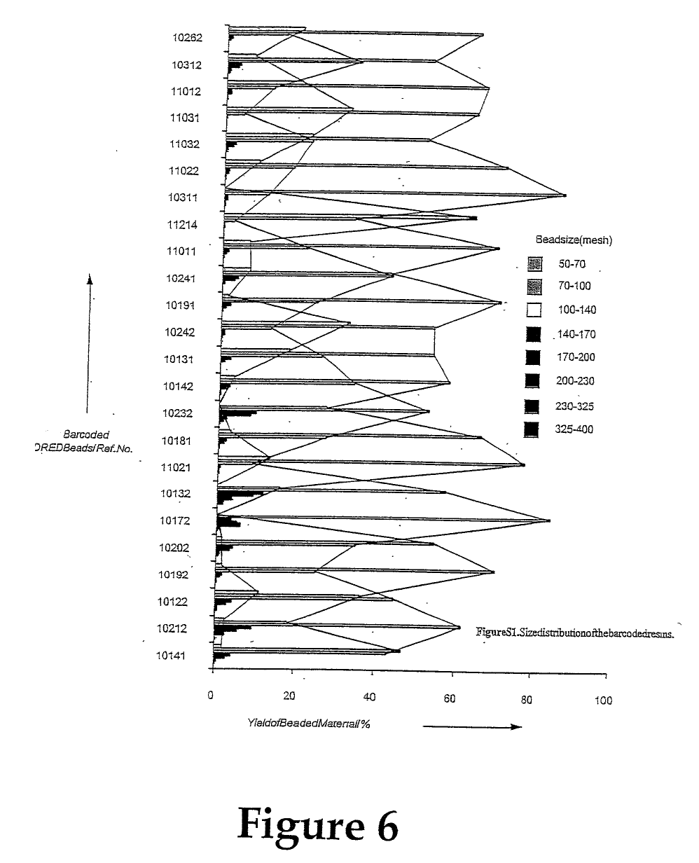

The micro-spherical beads were prepared by suspension co-polymerization following reported procedures as described by Arshady, R., Ledwith, A. React. Polym. 1983, 159-174. Typically, deionized water (200 mL), 10% (w/w) PVA/H₂O (4 g) were placed in the reaction vessel equipped with a mechanical stirrer, condenser and N₂ inlet. The reaction was kept under N₂ atmosphere throughout the entire polymerization process. An organic solution composed of co-monomers (for amounts see Table 1 of main text), DVB (0.125 g), chloromethylstyrene (0.50 g), BPO (0.15 g) was added to the reaction vessel. The mixture was stirred at a fixed speed (330 rpm) to produce the desired bead size distribution. The reactor was then immersed in a preheated oil bath maintained at 80° C. After 24 h, the motor was stopped and the beads formed were filtered and washed with deionized water and extracted with water and ethanol using Soxhlet extractors (24 h each). The beads were then sieved and dried under vacuum and characterized by FTIR and Raman spectroscopies, scanning electron microscopy and atomic force microscopy. FIG. 6 summarizes the bead size distribution of the 24 barcoded resins synthesized. Under the above suspension polymerization conditions the main fraction is in the range 70-140 mesh. FIGS. 5a and 5b show a typical scanning electron micrograph of the barcoded beads and an atomic force microscopy image of their surface morphology. The average surface roughness of the beads was determined to be 1.5 nm. Besides these surface fluctuations the AFM reveals randomly distributed holes with a depth of about 15 nm and a diameter in the range of 100 nm

Effect of the Dispersing Agent (Stabilizer) and Stirring Speed.

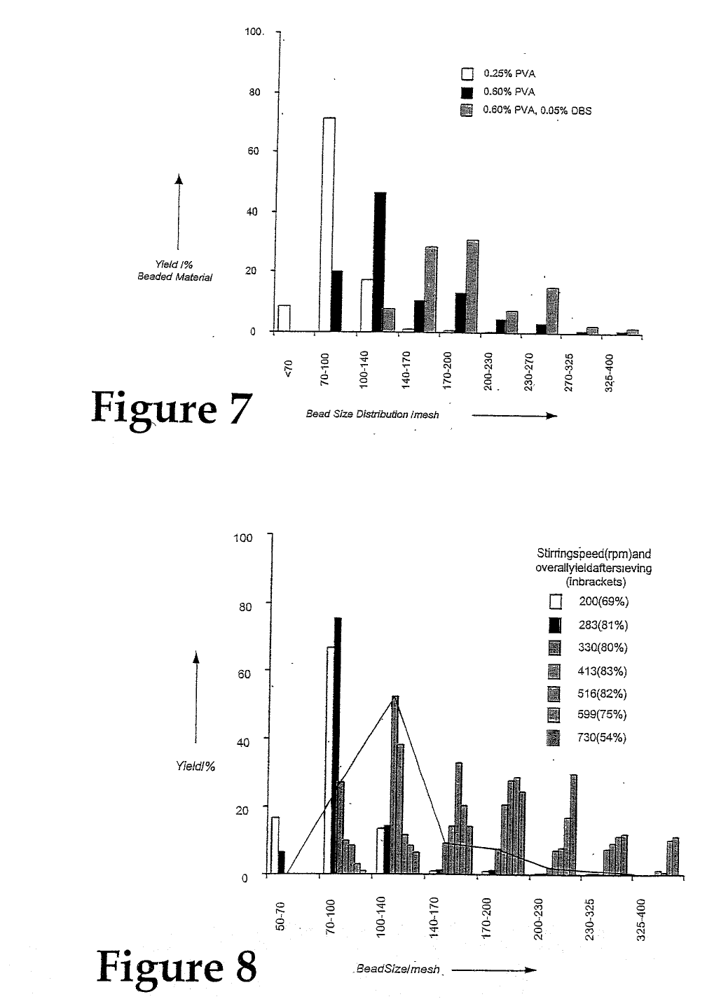

The synthesis yield and size distribution of the barcoded resins depend on several empirically determined parameters including the reactor design, the ratio of organic to aqueous phase, the rate of mixing (stirring speed), the viscosity of both phases, and on the concentration and chemical nature of the dispersing agent (stabilizer). The latter two parameters have the greatest impact on the suspension polymerization outcome and were, as a result, optimized first. During the suspension polymerization process the microdroplets formed are directly converted into microbeads, which may coagulate as their viscosity increases. To prevent this undesired aggregation, a stabilizer, usually a water-soluble polymer, is added. Some of the results obtained with PVA in the absence and presence of DBS are presented in FIG. 7. The effect of stirring speed on the bead size distribution and overall yield of beaded material (after sieving) is summarized in FIG. 8. Note that outside of the range 300-600 rpm the yield of beaded material drops steeply, and within this stirring range the distribution varies with the stirring rate. Based on these results the bead size distribution can be readily tuned to the desired size range.

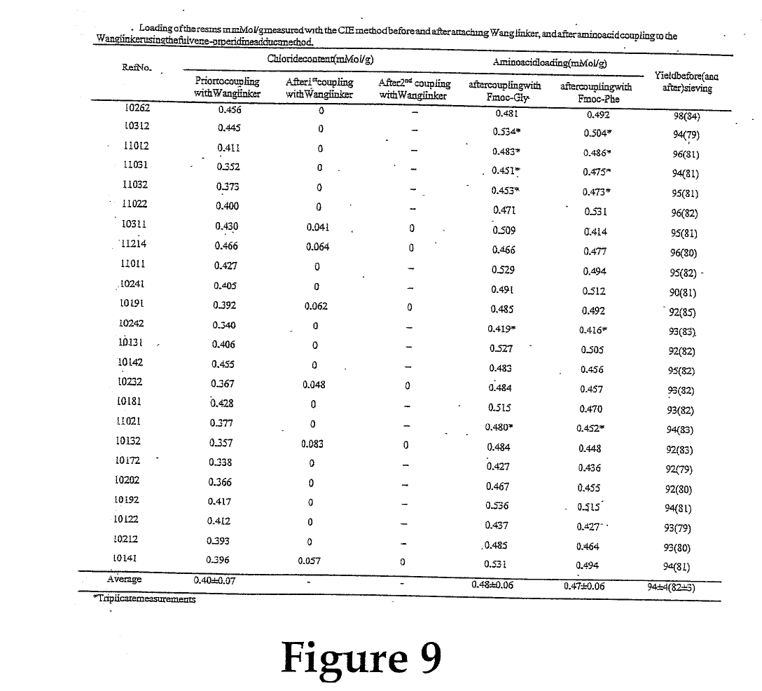

Chloromethylstyrene incorporation was quantified by potentiometric titration of the resins chloride content (FIG. 9) as described by Ma, t. et al., Modern Organic Elemental Analysis, Marcel Dekker Inc., New York, 1979., p. 164. The resin (0.05-0.1 g) and pyridine (1 mL) were sealed in a 10 mL glass vial and heated to 100° C. for 2 hr. the solution was then transferred to a 100 mL beaker and 50% HNO mL) was added. The potentiometric titration was carried out on an Orion 720A potentiometer equipped with an Orion IonPlus selective chloride ion electrode (CIE) and using a standard solution of AgNO₃ (0.0025725 M). The resins chloride content was derived from the following relationship:

$$C h l o r i d e s ( m M o l / g ) = \frac{V \times M}{w}$$

$$\begin{matrix} V = volume of {AgNO}_{3} added to reach the equivalence point \\ M = concentration of {AgNO}_{3} \\ W = weight of dry beads\end{matrix}$$

Attachment of the Wang linker to the Barcoded Beads.

The attachment of the Wang linker was accomplished as described in Wang, S. S. J. Am. Chem. Soc., 1973, 95, 1328-1333 and/or Lu, G. -S. et al. J Org. Chem. 1981, 46, 3433-3436. In particular, 4-(hydroxymethyl) phenol (134 mg), DMA (8 mL) and sodium methoxide (58 mg) were magnetically stirred under nitrogen in a 50 mL 2-necked round-bottom flask. After complete dissolution of the sodium methoxide (˜15 min), 800 mg of the resin were added and the mixture was heated to 50° C. in an oil bath for 8 hr. The beads were then washed sequentially with 1,4-dioxane (3×30 mL), distilled water/1,4-dioxane (1/1) (3×30 mL), 1,4-dioxane (3×30 mL), and methanol (3×30 mL).

The beads were then dried under high vacuum. The chloride content of the resin was measured as described above and when necessary a second coupling with the Wang linker was performed to cover the unreacted sites (FIG. 9).

Coupling with Fmoc-Gly and Fmoc-Phe.

In a peptide synthesis vessel, the resin beads (˜100 mg, 0.05 mMol hydroxyl groups based on potentiometric titration with a CIE) were soaked in DMF overnight then the solvent was drained and the resin washed with fresh DMF. A premixed solution of Fmoc-amino acid (200 μL of 0.5 M Fmoc-Gly in 1/1 DMF/DCM, or 400 μL of 0.25 M Fmoc-Phe in 1/1 DMF/DCM, 2 equiv.), DCC (400 μL of 0.5 M in 1/1 DMF/DCM, 4 equiv), and DMAP (200 1L, 0.05 M in 1/1 DMF/DCM, 0.2 equiv) were successively added to the resin. The volume was adjusted to 1 niL with DMF/DCM (1/1) and the slurry was shaken for 30 mm. The solvent was then drained and the procedure repeated twice. The solvent was removed and the resin washed thoroughly with DMF/DCM (1/1), DMF, DCM, and MeOH in this order (3×10 mL each).

The resin was then dried under high vacuum and the level of amino acid incorporation was determined by quantifying the fulvene-piperidine adduct (ε^{301 nm}=7,800 M⁻cm⁻¹) formed upon treatment with 20% piperidine/DMF (FIG. 9). This method yielded a somewhat higher average loading in comparison with the CIE titration method (048 0.06 mMol/g versus 0.40 0.07 mMol/g). This apparent discrepancy was attributed to the intrinsic nature of the CIE titration method, which involves the release of the chlorides from the resin via nucleophilic displacement with pyridine. A reaction of this nature may not take place in some of the hydrophobic pockets within the bead, thereby resulting in an apparently lower loading.

Swellability of the Beads.

The swelling properties of the beads prior to the attachment of the Wang linker were investigated in DMSO, THF, toluene, ethanol, DMF, DMA, DCM, and 1,4-dioxane (Table 2). The weighed beads were immersed in the solvent until swelling equilibrium was achieved (24 hr). Excess solvent was removed and the solvated beads were weighed. The swelling degree was then derived from the following equation:

$$S w e l l a b i l i t y ( mL / g ) = \frac{( \frac{{W}_{sol} - {W}_{d}}{d}}{{W}_{d}}$$

$$\begin{matrix} {W}_{sol} = solvated wight of the beads \\ {W}_{d} = dry weight of the beads \\ {d}_{s} = density of the solvent\end{matrix}$$

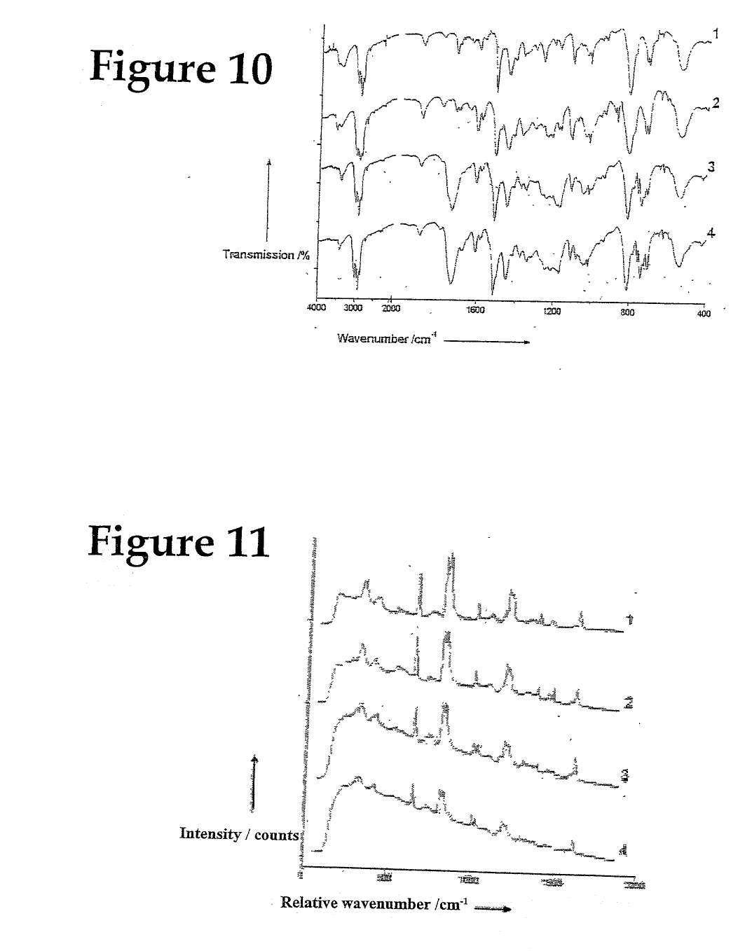

Effect of the Wang Linker and amino acids on the resins Raman and IR barcodes was determined. FIGS. 10 and 11 show that the linker and amino acids have no effect on the main features of the IR and Raman spectra of the barcoded beads. (1) Bead #10241; (2) bead #10241+Wang linker; (3) bead #10241+Wang linker +Fmoc-Gly; (4) bead #10241+Wang linker +Fmoc-Phe.

The foregoing discussion discloses and describes merely exemplary embodiments of the present invention. One skilled in the art will readily recognize from such discussion, and from the accompanying drawings, that various changes, modifications and variations can be made therein without departing from the spirit and scope of the invention.

All references cited herein are expressly incorporated by reference.

1. A polymeric resin comprising at least one co-monomer, wherein said co-monomer has a unique spectra and wherein the polymeric resin has a unique spectral fingerprint.

2. The polymeric resin of claim 1 wherein the unique spectra is a Raman spectra.

3. The polymeric resin of claim 1 wherein the unique spectra is an infared spectra.

4. The polymeric resin of claim 1 wherein the co-monomer is a styrene-based co-monomer.

5. The polymeric resin of claim 4 wherein the styrene-based co-monomer is selected from the group consisting of styrene, 2,5-dimethylstyrene, 4-methylstyrene, 2,4-dimethylstyrene, 4-tert-butylstyrene and 3-methylstyrene.

6. The polymeric resin of claim 1 wherein the polymeric resin comprises at least two different co-monomers.

7. The polymeric resin of claim 1 further comprising a reactive co-monomer, wherein the amount of reactive co-monomer does not contribute to the spectral fingerprint of the polymeric resin.

8. The polymeric resin of claim 7 further comprising a chemical linker covalently bound to the reactive co-monomer.

9. The polymeric resin of claim 8 wherein the chemical linker is 4-hydroxymethylphenol.

10. A method for determining the primary structure of a first compound which is bound to the polymeric resin of claim 8 comprising the steps of:
(a) reacting a first building block of said first compound with said polymeric resin;
(b) subjecting said first solid support matrix to a spectroscopic technique so as to generate spectral data of said polymeric resin;
(c) determining a chemical composition of said polymeric resin based upon said data generated by said spectrographic technique; and determining the chemical identity of said first building block based upon the chemical composition of said polymeric resin.

11. The use of the resin of claim 8 in dual recursive deconvolution in generating and screening a combinatorial library.

12. A method for determining the primary structure of a first compound which is bound to a polymeric resin comprising the steps of:
(a) reacting a first building block of said first compound with said polymeric resin, wherein said polymeric resin comprises at least one co-monomer, wherein said co-monomer has a unique spectra and wherein the polymeric resin has a unique spectral fingerprint;
(b) subjecting said first solid support matrix to a spectroscopic technique so as to generate spectral data of said polymeric resin;
(c) determining a chemical composition of said polymeric resin based upon said data generated by said spectrographic technique; and determining the chemical identity of said first building block based upon the chemical composition of said polymeric resin.

13. The method of claim 12 wherein the spectroscopic technique is Raman spectroscopy.

14. The method of claim 12 wherein the spectroscopic technique is infared spectroscopy.

15. The method of claim 12 further comprising generating a combinatorial library after step (a) and prior to step (b).

16. A method of determining the structure of a compound which is bound to a solid support matrix, comprising:
(a) subjecting said solid support matrix to a spectroscopic technique so as to generate spectrographic data of said solid support matrix;
(b) determining a chemical composition of said solid support matrix based upon said spectrographic data generated; and
(c) determining the chemical identity of a building block of said compound based upon said chemical composition of said solid support matrix.

17. The method of claim 16, wherein:
(a) includes subjecting said solid support matrix to an infrared spectroscopic technique.

18. The method of claim 16, wherein:
(a) includes subjecting said solid support matrix to a Raman spectroscopic technique.

19. The method of claim 16, wherein:
(a) includes subjecting said solid matrix to electromagnetic radiation having a wavenumber from about 400 cm−1 to about 2000 cm−1.

20. The method of claim 16, wherein:
said solid support includes a co-monomer selected from the group consisting of 2,5-dimethylstyrene, 4-methylstyrene, 2,4-dimethylstyrene, 4-tert-butylstyrene and 3-methylstyrene.
